(19) United States
(12) Patent Application Publication
(10) Pub. No.: US 2010/0108071 A1
(43) Pub. Date: May 6, 2010

(54) APPARATUSES TO FILTER AIR

(76) Inventor: Bradford Macy, JR., Concord, CA (US)

Correspondence Address: IVY Y. MEI, 800 EL CAMINO REAL W., SUITE 180, MOUNTAIN VIEW, CA, 94040, US

(21) Appl. No.: 12/609,014

(22) Filed: Oct. 30, 2009

Related U.S. Application Data
(60) Provisional application No. 61/197,997, filed on Oct. 30, 2008.

(51) Int. Cl. A62B 23/06; A61M 16/00

(52) U.S. Cl. 128/206.11; 128/205.27

(57) ABSTRACT

Apparatuses for personal air filtering. In one aspect, a personal air filtering system includes nasal plugs to be inserted into the nostrils and a main body worn between the nose and mouth. In another aspect, the personal air filtering system has a housing that prevents exhaled air from entering the air filter disposed in the housing. In a further aspect, the personal air filtering system has separate vents for inhalation and exhalation.

### RELATED APPLICATIONS

The present application claims priority to the provisional U.S. Patent Application Ser. No. 61/197,997, filed Oct. 30, 2008 and entitled “Nasal Air Filter”, the disclosure of which is hereby incorporated herein by reference.

### FIELD OF THE TECHNOLOGY

At least some embodiments of the disclosure relate to filters in general and, more particularly but not limited to, air filtering systems.

### BACKGROUND

Particulate air pollution has been shown to cause significant increases in the incidence of cardiopulmonary and respiratory diseases, such as asthma, bronchitis, and lung cancer.

Particulate air pollution is typically a mixture of solids and liquids. Particles come in a wide range of sizes. Those less than 10 micrometers in diameter (PM10) are so small that they can get into the lungs, potentially causing serious health problems. Particles less than 2.5 micrometers in diameter are known as “fine” particles. Some sources of fine particles include various types of combustion, including motor vehicles, power plants, residential wood burning, fires, agricultural burning, and some industrial processes.

Particles between 2.5 and 10 micrometers in diameter are referred to as “coarse.” Sources of coarse particles include crushing or grinding operations, and dust stirred up by vehicles traveling on roads.

Although particulate pollution affects all individuals and can increase the risk of respiratory illness, people sensitive to pollution, such as asthmatics, and those with heart conditions or lung diseases, may experience distress and other health effects, even at lower levels of pollution than the average person.

More than 150 million people suffer from asthma in the world and the number has been rising steadily since the 1980s. In the last 10 years, asthma cases have risen 50% worldwide. The countries which are worst affected by asthma are the USA, Ireland, the UK, Australia and New Zealand.

The statistics on asthma in the United States are staggering. Approximately 22 million Americans currently have asthma. More than 70% of these people also suffer from allergies. The prevalence of asthma increased 75% from 1980 to 1994 and is still increasing. Asthma rates for children under the age of five increased 160% between 1980 and 1994. Approximately 5000 people die directly related to asthma each year in the United States.

The direct health care costs for asthma in the U.S. total more than $11.5 billion annually and indirect costs account for another $4.6 billion. Direct costs include clinic and emergency room visits, medications, and hospitalizations. In 2003 the total days missed in school by children due to asthma was 12.8 million and the number of missed work days was 11.8 million.

Statistics in the United Kingdom are even worse. Some studies in the UK have suggested that particulates are responsible for up to 10,000 premature deaths in that country each year. More than five million persons in the United Kingdom suffer from Asthma. This represents more than 15% of the total population and 20,000 new cases per month are being reported! According to a study by The British Thoracic Society, child asthma rates have quadrupled since 1973 when rates were 5.5% of children. This rate increased to 27% in 2003. In Australia, one child in 3 under 16 has the asthma symptoms.

Many different types of masks exist which are used to filter varying degrees and sizes of particulate air pollution. However, most people are resistant to wearing masks as they are uncomfortable to wear, and restricting around the face. In addition, most masks get warm and damp after a short use. Many individuals with respiratory disease who would greatly benefit from filtering the air they are breathing refuse to wear masks because they are uncomfortable and do not last very long. Even healthy people who exercise or spend their leisure time outdoors in bad air quality do not wear masks for the reasons outlined above.

Masks used for biological protection such as National Institute for Occupational Safety and Health (NIOSH) certified N95 respirators can be difficult to fit, because such a large surface area of the face needs to be completely sealed. Facial hair can inhibit seal formation. It is difficult to protect the general public from disease outbreak with such a mask, as each person must be fit tested and trained in use.

### SUMMARY OF THE DESCRIPTION

Apparatuses for personal air filtering are disclosed herein. Some embodiments are summarized in this section.

In one aspect, a personal air filtering system includes nasal plugs to be inserted into the nostrils and a main body worn between the nose and mouth. In another aspect, the personal air filtering system has a housing that prevents exhaled air from entering the air filter disposed inside the housing. In a further aspect, the personal air filtering system has separate vents for inhalation and exhalation.

In one aspect, an apparatus includes: an air filter; and a housing to hold the air filter inside the housing, the housing having at least a first vent to allow air to enter inside the housing through the air filter and at least a second vent to allow air to exit from inside of the housing. The housing prevents exhaled air from going through the air filter. In one embodiment, the apparatus further includes: two plugs coupled with the housing and adapted to be plugged into nostrils of a person to provide air passages between respective nasal passages of the person and inside of the housing; and two hooks coupled with the housing and adapted to be supported by ears of the person while the plugs are plugged into the nostrils of the person.

In one embodiment, the housing defines: a first compartment defining an interior space connected to interiors of the plugs to provide the air passages; and a second compartment to hold the air filter between the at least first vent and the first compartment. For example, the housing may further includes a first wall to separate the first compartment and the second compartment; and a first valve disposed on the first wall to prevent air flow between the first compartment and the second compartment when the person exhales and to allow air flow between the first compartment and the second compartment when the person inhales.

In one embodiment, the housing further defines: at least one third compartment defining at least one interior space connected to openings of the at least one second vent. For example, the housing may further include: at least one second wall to separate the first compartment and the at least one third compartment; and at least one second valve disposed on the at least one second wall to prevent air flow between the first compartment and the at least one third compartment when the person inhales and to allow air flow between the first compartment and the at least one third compartment when the person exhales.

In one embodiment, the air filter includes a pre-filter and a HEPA accordion shaped filter. In one embodiment, the air filter further includes a carbon filter disposed between the pre-filter and the HEPA accordion shaped filter.

In one embodiment, the housing includes a cover; and the air filter is replaceable when the cover is removed or detached.

In one embodiment, the apparatus further includes: an electronic device to provide audio signals; and earphones coupled to the electronic device and supported by the hooks. In one embodiment, the electronic device includes a music player.

In another aspect, an apparatus includes: a housing to hold an air filter inside the housing; and two plugs disposed on the housing and adapted to be plugged into nostrils of a person to provide air passage between respective nasal passages of the person and outside of the housing through the air filter held inside the housing.

In one embodiment, the housing is configured to be disposed between the nose of the person and the mouth of the person. The apparatus may further include ear hooks coupled to the housing to provide support via the ears of the person.

In one embodiment, the housing does not cover the face of the wearer above the nose of the person from outside of the nose and does not cover the mouth of the person when the person opens the mouth.

In one embodiment, each of the two plugs includes: a tube to provide an air passage from a nasal passage of the person to inside of the housing; and compressible foam to air-tightly engage the plug with interior of a nostril of the person.

In one embodiment, the housing includes an upper exterior wall and a lower exterior wall; the plugs are disposed over the upper exterior wall; and when the plugs are inside the nostril of the person, the upper exterior wall is under the nose of the person and the lower exterior wall above the mouth opening of the person.

In one embodiment, the housing includes a removable cover, which when removed provides an opening to service the air filter. In one embodiment, the housing includes separate exhalation vents and inhalation vents.

In one embodiment, the apparatus further includes an air filter housed within the housing.

Other features will be apparent from the accompanying drawings and from the detailed description which follows.

### BRIEF DESCRIPTION OF THE DRAWINGS

The embodiments are illustrated by way of example and not limitation in the figures of the accompanying drawings in which like references indicate similar elements.

FIG. 1 shows a view from an upper side of a personal air filtering system according to one embodiment.

FIG. 2 shows a view from a lower side of a personal air filtering system according to one embodiment.

FIG. 3 shows a view of a personal air filtering system with a housing cover being separate from the main body according to one embodiment.

FIG. 4 shows a view from an upper side of a personal air filtering system with a housing cover being removed from the main body according to one embodiment.

FIG. 5 shows a view from an upper side of a personal air filtering system with a housing cover and a filter cartridge being removed from the main body according to one embodiment.

FIG. 6 shows a segment of a personal air filtering system according to one embodiment.

FIG. 7 shows a segment of a personal air filtering system with a filter cartridge being removed according to one embodiment.

FIG. 8 illustrates an inhalation air flow path through a segment of a personal air filtering system according to one embodiment.

FIG. 9 illustrates an exhalation air flow path through a segment of a personal air filtering system according to one embodiment.

FIG. 10 illustrates a cross section of a nasal plug according to one embodiment.

FIG. 11 illustrates a usage of a personal air filtering system according to one embodiment.

### DETAILED DESCRIPTION

The following description and drawings are illustrative and are not to be construed as limiting. Numerous specific details are described to provide a thorough understanding. However, in certain instances, well known or conventional details are not described in order to avoid obscuring the description. References to one or an embodiment in the disclosure are not necessarily references to the same embodiment; and, such references mean at least one.

One embodiment of the disclosure provides a personal air filtering system for the comfortable breathing of filtered air through the nasal passages.

In one embodiment, a personal air filtering system can filter the air in several different ways including, but not limited to High Efficiency Particulate Air (HEPA) filtration, and carbon filtration. Unlike a mask, which covers both the mouth and nose, the personal air filtering system plugs only the nasal passages, allowing the face to be exposed and making it easy to talk, eat and drink. Wearing such a personal air filtering system would allow an individual better social interaction than if they were wearing any type of existing masks.

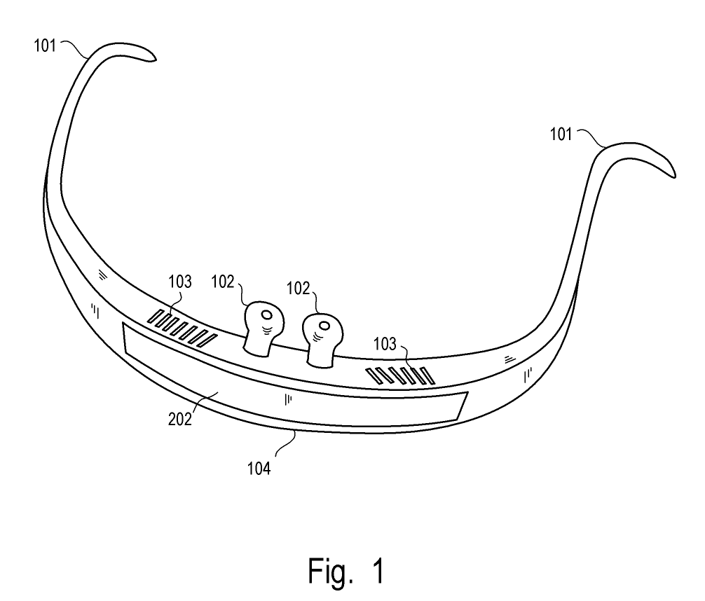

FIG. 1 shows a view from an upper side of a personal air filtering system according to one embodiment. In FIG. 1, the personal air filtering system includes a main body (104), two ear hooks (101) and two nasal plugs (102).

In one embodiment, the personal air filtering system can be attached to the ears of a person with the ear hooks (101), like a pair of glasses. The nasal plugs (102) fit into the nostrils of the person, blocking air from going into the nose without first going through the main body (104) and being filtered.

In FIG. 1, the upper exterior wall of the main body (104) has vents (103). In one embodiment, the vents (103) on the upper exterior wall of the main body (104) are provided to allow exhaled air to leave the main body (104).

In FIG. 1, a housing cover (202) is disposed on the front side of the main body (104). An air filter cartridge is disposed inside the main body (104), behind the housing cover (202). In one embodiment, the housing cover (202) is removable/detachable for the replacement of the air filter cartridge.

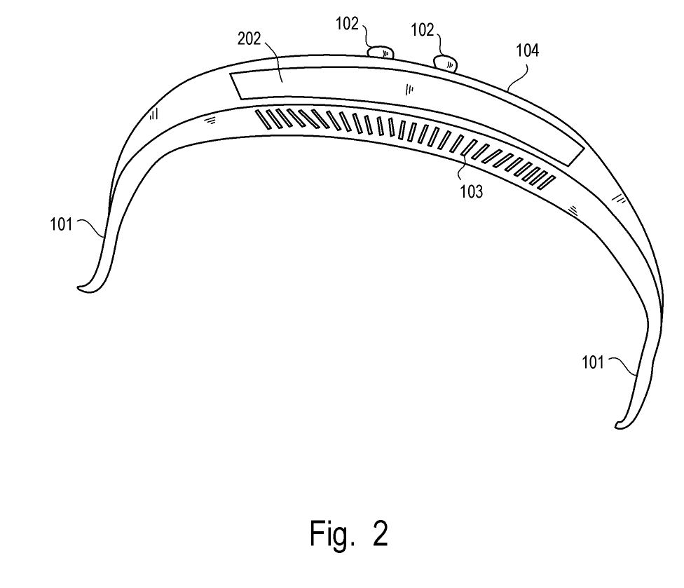

FIG. 2 shows a view from a lower side of a personal air filter system according to one embodiment. In FIG. 2, the lower exterior wall of the main body (104) has vents (103). In one embodiment, the vents (103) on the lower side of the personal air filter are provided to allow air to enter the main body during inhalation. The air enters the main body through the vents (103) on the lower exterior wall of the main body (104), passing the air filter cartridge located behind the housing cover (202) to enter the nasal passages through the nasal plugs (102).

In one embodiment, the ear hooks (101) serve to support and/or hold the distal ends of the personal air filtering system on the wearers face. For example, the ear hooks (101) can be made of a plastic body with a metal wire running through the plastic body, which allows bending to adjust the angle. In other embodiments, the ear hooks (101) may be made of other materials, or may have other adjustable characteristics to be adapted to the configuration of the head of a particular user.

In some embodiments, the body of the personal air filtering system may be supported by a strap around the head, with or without the ear hooks (101). In other embodiments, the personal air filtering system may simply be held in place by the nasal plugs (102).

In one embodiment, the nasal plugs (102) serve to block air from entering the nostrils without first going into the personal air filtering system and being filtered. Air travels by way of least resistance, through the personal air filtering system and delivers filtered air to the wearer.

In one embodiment, the main body (104) of the personal air filtering system is made of a plastic structure which is slightly curved to fit the contour of the face of a person at the upper lip area extending around the upper jaw, but the main body (104) could be made of other light weight materials such as composite or metals.

In one embodiment, the main body (104) is about 5″×0.75″×0.5″, but could be larger or smaller in any dimension, or could be made in different sizes to fit different users facial dimensions.

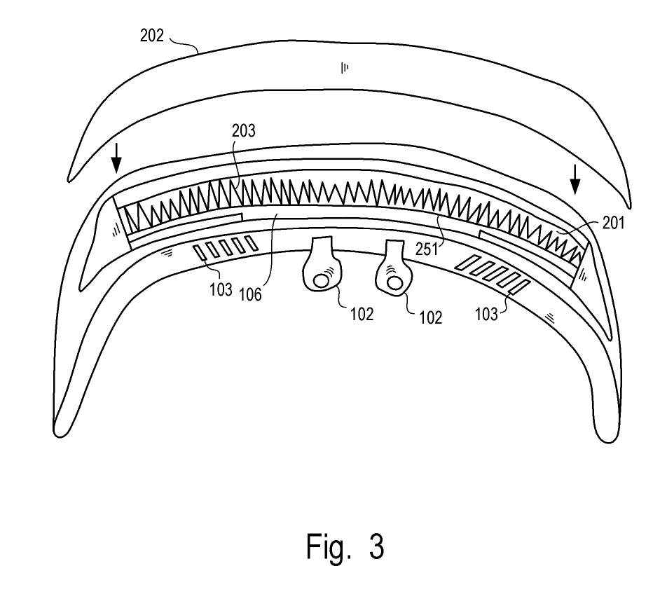

FIG. 3 shows a view of a personal air filtering system with a housing cover being separate from the main body according to one embodiment. In FIG. 3, the air filter cartridge (203) is disposed within the filter housing (201). In one embodiment, the air filter cartridge (203) is disposable. After the house cover (202) is detached or removed from the main body (104), the used air filter cartridge (203) can be removed and discarded; and a new, fresh air filter cartridge (203) can be inserted in the filter housing (201). After the housing cover (202) is attached back to the main body (104), the personal air filtering system can be again used.

In FIG. 3, a wall (251) separates the filter housing (201) and the air transfer chamber (106). In one embodiment, the incoming air filtered through the filter cartridge (203) is allowed to enter the air transfer chamber (106) (e.g., via one or more one way valves on the wall (251)). Exhaled air from the nasal plugs (102) can enter the air transfer chamber (106), but cannot flow back to the filter housing (201) (e.g., being blocked by the one or more one way valves during exhalation). The exhaled air is allowed to escape from the vents (103) (e.g., located on the upper exterior wall of the main body (104) as illustrated in FIG. 1), separate from the vents (103) that take the incoming air (e.g., vents located on the lower exterior wall of the main body (104) as illustrated in FIG. 2).

In one embodiment, the back and side walls of the main body (104) are solid plastic and air tight. The front wall includes a removable housing cover (202) which snaps into place to form an air tight compartment for the filter cartridge (203). In other embodiments, the housing cover (202) may be attached to the main body (104) via other means, such as screw, glue, sliding slot, etc.

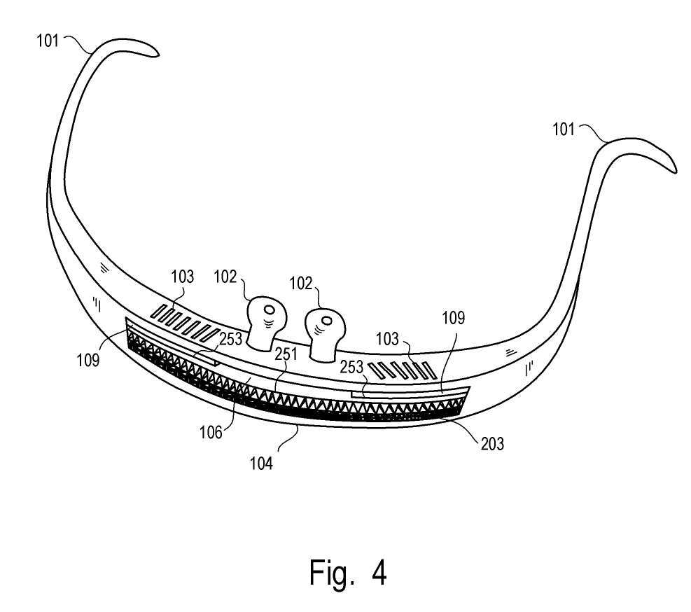

FIG. 4 shows a view from an upper side of a personal air filtering system with a housing cover being removed from the main body according to one embodiment. In FIG. 4, the air transfer chamber (106) and the exhalation compartments (109) are separate by walls (253). One way valves between the air transfer chamber (106) and the exhalation compartments (109) allow air to flow out from the air transfer chamber (106) to the exhalation compartments (109) and then exit from the exhalation vents (103) shown in FIG. 4. However, the one way valves between the air transfer chamber (106) and the exhalation compartments (109) prevents the air from entering the air transfer chamber (106) via the exhalation compartments (109). Thus, the main body (104) allows the exhaled air to exit from the main body without going through the air filter cartridge (203).

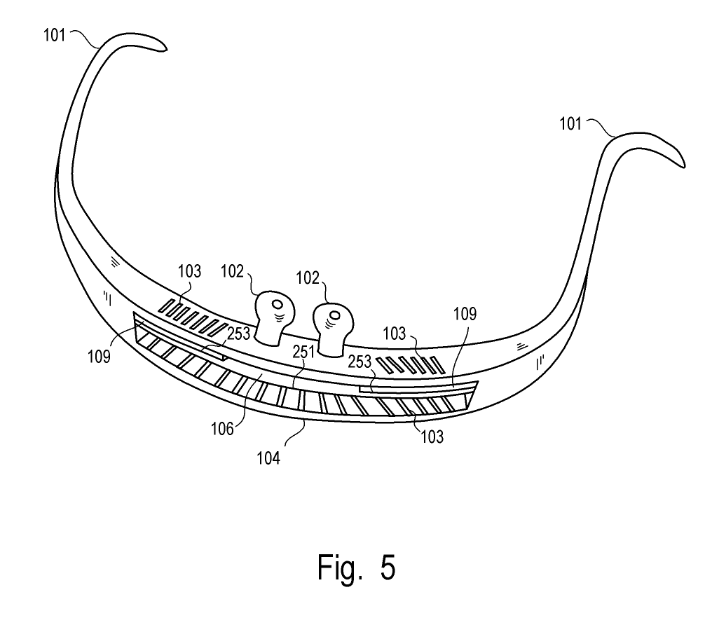

FIG. 5 shows a view from an upper side of a personal air filtering system with a housing cover and a filter cartridge being removed from the main body according to one embodiment. In FIG. 5, the inhalation vents (103) are located at the lower exterior wall of the main body (104), visible through the opening in the main body (104) when the housing cover (202) is removed or detached. The incoming air from the inhalation vents (103) would go through the air filter cartridge (203) as illustrated in FIG. 4 to enter the air transfer chamber (106) and then flow through the passages inside the nasal plugs (102) into the nasal passages of the wearer.

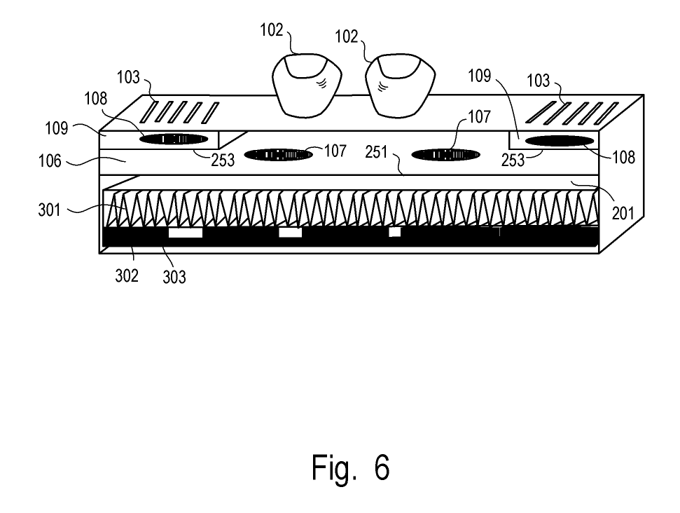

In one embodiment, the wall (251) that separates the air filter housing (201) and the air transfer chamber (106) has one or more one way valves to prevent expired, exhaled air from entering back into the air filter housing (201), as illustrated in FIG. 6.

FIG. 6 shows a segment of a personal air filtering system according to one embodiment. In FIG. 6, the nasal plugs (102) are seated on the superior aspect of the main body (104), with the exhalation vents (103) on either side of the nasal plugs (102).

In FIG. 6, a wall (253) separates the exhalation vents (103) from the air transfer chamber (106). The cavity produced there is the exhalation compartment (109). Expired air travels into the air transfer chamber (106), through the exhalation valves (108), into the exhalation compartment (109) and out through the exhalation vents (105).

In one embodiment, the air filter cartridge (203) includes a cotton pre-filter (303), a carbon filter (302) and a HEPA accordion shaped filter (301). Other types of air filter cartridges can also be used. In some embodiments, the filter cartridge (203) may be integrated with the housing (201), and the entire personal air filtering system may be disposable. In other embodiments, there may be a disposable prefilter cartridge and a filter cartridge separate from the prefilter. In further embodiments, a filter portion may be coupled to a support portion to form the personal air filtering system; and the filter portion may include the filter cartridge (203) and/or the nasal plug (102) and may be removable and disposable.

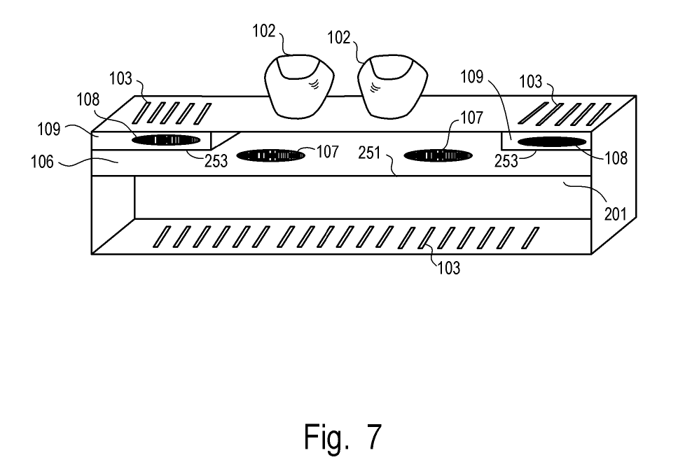

In one embodiment, the inhalation vents (103) are found on the inferior surface of the main body (104), as illustrated in FIG. 7.

FIG. 7 shows a segment of a personal air filtering system with a filter cartridge being removed according to one embodiment. In FIG. 7, the filter housing (201) is empty; and the inhalation vents (103) can be seen on the inferior surface of the main body (104).

In FIG. 7, the filter housing (201) is constructed to allow air flow from inhalation vents (103) at the inferior portion of the main body (104) through the filter cartridge (203) and the inhalation valves (107), disposed on the wall (251) located on the superior portion of the filter housing (201), to air transfer chamber (106) and then to the passages inside the nasal plugs (102).

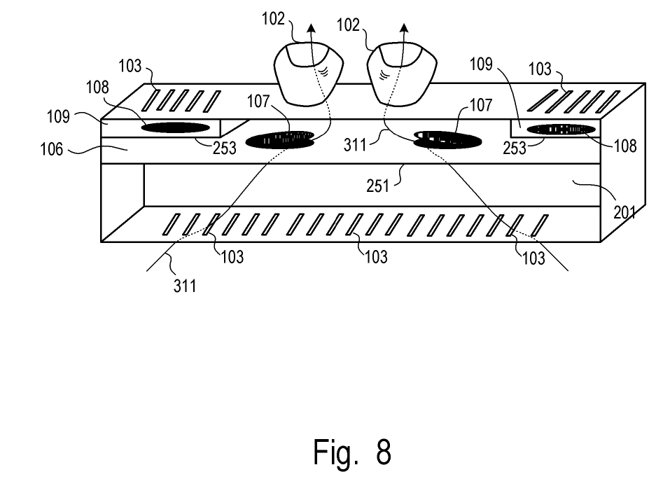

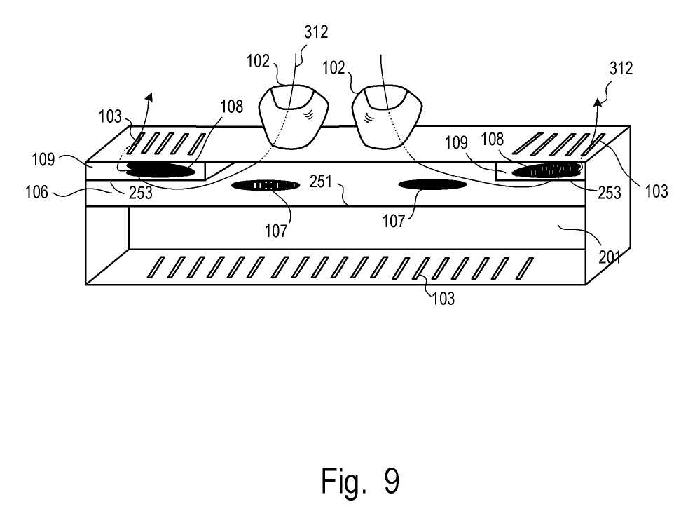

In one embodiment, the air transfer chamber (106) is a small compartment formed above the filter housing (201). The air transfer chamber (106) is configured to receive both inhaled and exhaled air. The inhalation valves (107) are disposed on the wall (251) located on the floor of the air transfer chamber (106) and open when negative pressure is created in the air transfer chamber (106) by inhalation of air by the lungs. The exhalation valves (108) are forced shut during inhalation by negative pressure and/or tension spring action, and open when positive pressure is created in the air transfer chamber (106) during exhalation by the lungs, as further illustrated in FIGS. 8 and 9.

FIG. 8 illustrates an inhalation air flow path (311) through a segment of a personal air filtering system according to one embodiment. In FIG. 8, the inhalation causes the air pressure within the air transfer chamber (106) to be lower than ambient air pressure. Thus, the inhalation valves (107) open to allow the air from outside of the main body (104) to enter through the inhalation vents (103) formed at the inferior portion of the main body (104), pass through the filter housing (201) and reach the air transfer chamber (106); and the exhalation valves (108) close to prevent unfiltered air from entering into the air transfer chamber (106) via the exhalation vents (103) formed on the superior portion of the main body (104).

FIG. 9 illustrates an exhalation air flow path (312) through a segment of a personal air filtering system according to one embodiment. In FIG. 9, the exhalation causes the air pressure within the air transfer chamber (106) to be higher than ambient air pressure. Thus, the inhalation valves (107) close to prevent the expired/exhaled air from entering the filter housing (201) from the air transfer chamber (106); and the exhalation valves (108) open to allow the expired/exhaled air to flow from the air transfer chamber (106) to the exhalation compartments (109) and to exit the main body (104) via the exhalation vents (103) formed on the superior portion of the main body (104).

In one embodiment, the filter housing (201) has a cavity in the center of the main body within which the disposable filter cartridge (203) is placed. For example, the filter housing is 4″×0.5″×0.4″ but may be different sizes or dimensions in other embodiments.

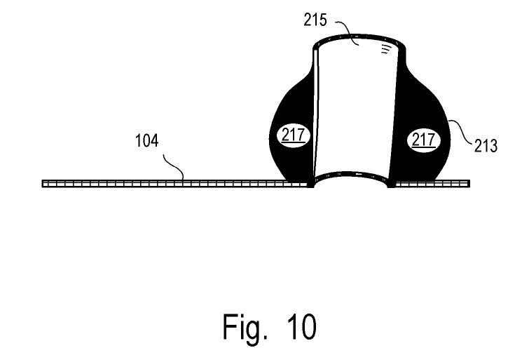

FIG. 10 illustrates a cross section of a nasal plug according to one embodiment. In FIG. 10, the nasal plug (102) can be made of disposable soft foam (217) which expands after being squeezed and placed into the nostrils. Once they re-expand, they form an airtight seal within the nostrils. In one embodiment, the nasal plug (102) includes a liner (213), which may be made of latex or other pliable, non-absorbent material.

In FIG. 10, the nasal plug (102) is formed via a tube that provides an air passage (215) from the air transfer chamber (106) in the main body (104) to the sinus of the wearer.

Other embodiments of the nasal plugs (102) could include, but are not limited to, the use of latex or silicone, both of which are soft, flexible and bio-safe. The nasal plugs (102) can be inflated to fill the nostrils and block air from bypassing the filter, but in other embodiments, inflation may not be necessary.

In some embodiments, at least part of the nasal plugs (102), such as the liner (213) and/or the foam (217), is removable, detachable from the main body (104). Thus, used parts of the nasal plugs (102) may be replaced with fresh ones for improved user experience.

In other embodiments, the main body (104) of the personal air filtering system may be different shapes or sizes and/or have a port to hook up oxygen. It could have different designs or styles, for which the wearer picks a style to fit his or her persona.

In one embodiment, the personal air filtering system includes an ear phone and microphone attachment to use as hands free wireless device, or mp3, or any other portable electronic devices. For example, the support portion outside the main body (104) may include an electronic device, such as MP3 player, a radio receiver, a micro phone, etc. The support portion, including the ear hooks (101), may also include a pair of ear phones. The electronic device may connect to other devices via wireless connections, such as Bluetooth connections.

In one embodiment, the personal air filtering system has a sensor that communicates to the user by sound or light when the filter is due to be changed.

In some embodiments, the inhalation vents (103) may be positioned at other locations, such as the side or the front of the device. Alternatively or in combination, the exhalation vents (103) may also be positioned at other locations, such as the front, the side or the bottom of the device.

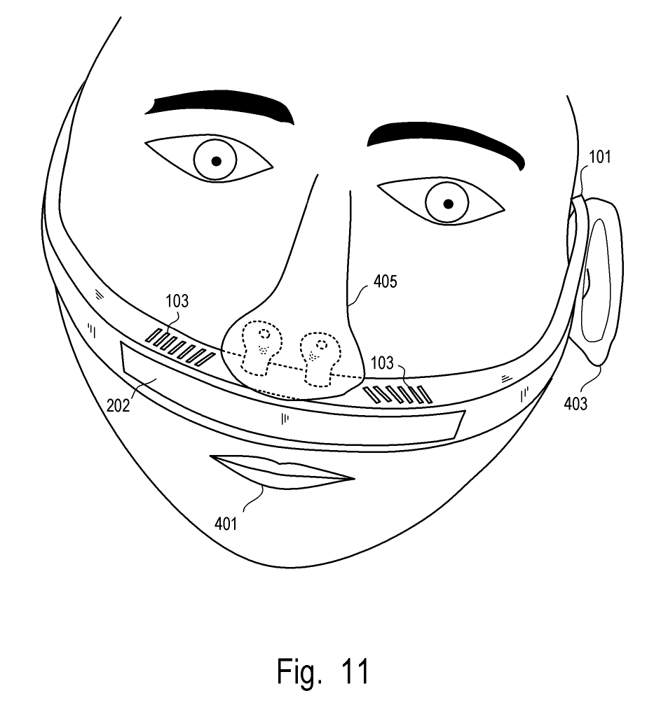

In one embodiment, the main body (104) of the device is configured to be worn between the nose and the mouth, as illustrated in FIG. 11.

FIG. 11 illustrates a usage of a personal air filtering system according to one embodiment. As illustrated in FIG. 11, the nasal plugs (102) are inserted in the nostrils of the nose (405); and the ear hooks (101) are supported on the ears (403). The main body (104) is above the mouth (401). The upper exterior wall of the main body has the exhalation vents (103) and is level and under the nose. The lower exterior wall of the main body has the inhalation vents and is level and above the lower lip.

In one embodiment, air enters the main body (104) through inhalation vents (103) (as illustrated in FIGS. 2, 5 and 8) on the lower aspect of the main body (104) of the unit. This air runs into the filter housing (201) and through a disposable filter unit (203) within the housing (201). From here the filtered air goes through two one way inhalation valves (107) on the upper wall (251) of the filter housing (201) and into the air transfer chamber (106). From here it enters the nasal plugs (102) and is inhaled into the nose (405).

In one embodiment, expired/exhaled air travels from the nostrils though the nasal plugs (102) and into the air transfer chamber (106). Positive pressure in the air transfer chamber (106) closes the one way inhalation valves (107) on the top wall (251) of the filter housing (201) to prevent expired air, which is moist and may be contaminated, from entering the filter housing (201). At the same time, the positive pressure opens the exhalation valves (108) located on the floor (253) of each exhalation compartment (109), allowing exhaled air to exit into the corresponding exhalation compartment (109) and then through the exhalation vents (as illustrated in FIGS. 1, 5 and 9).

In other embodiments, the valves (107 and 108) may close by means of low tension springs and, or positive or negative pressure.

In one embodiment, the entire device is easy to clean either with alcohol or soap and water. The wearer would simply remove the disposable filter cartridge from the filter housing and remove the disposable part of the nasal plugs (102). The body of the device and housing cover could then be immersed in an appropriate cleaning solution and the device would be left out to dry.

In other embodiments, the main body (104) of the device is configured to be partially worn above the nose.

In some embodiments, exhalation valves (108) are used without inhalation valves (107). The part of the exhaled air may go through the filter housing (201).

In some embodiments, inhalation valves (107) are used without exhalation valves (108). For example, the exhalation compartment (109) may include an air filter to prevent unfiltered air to enter the air transfer chamber (106) and allow exhaled air to exit from the exhalation vents (103).

In some embodiments, neither inhalation valves (107) nor exhalation valves (108) are used. For example, both inhaled air and exhaled air may be forced to go through the filter housing (106).

In some embodiments, the filter (203) is integrated with the nasal plugs (104) as a disposable component.

The personal air filtering systems according to various embodiments disclosed herein solve many of the problems associated with masks. For example, in one embodiment, the personal air filtering system is not restricting and moisture does not build up, as exhaled air is vented out separate from inhaled air.

Because the personal air filter system only needs to seal the nostrils, it would be easy to use to protect against disease outbreak or bio-terror among the general public.

The device would be desirable for athletes who want to breathe filtered air while exercising and for anyone exposed to poor air quality who wants to breathe healthy air. It could protect the wearer from particulate pollution and allergens allowing for healthier exercise.

Asthmatics and other persons with pulmonary conditions for whom exposure to particulate air pollution can have serious adverse effects. For instance, individuals with respiratory illness, such as COPD, asthma, and lung cancer, breathing polluted air is particularly harmful. For these individuals, the personal air filtering system could greatly improve their quality and possibly the longevity of life.

Oxygen can be run right through the main body of the personal air filtering system.

Persons with allergies can have mild to serious reactions to certain allergens in the environment. The quality of life for these individuals could improve greatly with the use of the personal air filtering system. For example, they may be able to rely less heavily on allergy drugs which can be drying, sedating, and expensive. For example, wearing the filter could allow persons to go places and do things that they previously could not, such as, visiting friends with pets, or going to the park in the springtime. For example, the control of allergies means the control of inflammation which means an overall healthier person.

Other types of persons may also benefit from the use of the personal air filtering systems disclosed herein, such as immune suppressed individuals who need to filter the air they breathe, workers in factories, refineries, bridge toll workers, highway workers, construction workers, miners, or any person who is constantly exposed to particulates in their line of work. Also, soldiers who are exposed to smoke, dust, and other toxic particulates including the possibility of biological and some chemical weapon attacks, and health care workers working with patients in isolation. Health care workers could offer more personal care with their face exposed than having it covered with a mask. It is also more comfortable and provides a safer seal against biological pathogens.

Further, the personal air filtering systems can be used by the general public in times of disease outbreak, biological terror, or in crowded areas to avoid common infections such as colds or flu, or less common infections such as pneumonias and tuberculosis, or when they are in areas where particulate pollution is problematic and they want to decrease their exposure for health or comfort reasons. Examples are in big cities where air pollution and cigarette smoke is a problem, or in areas where large fires are burning.

In the foregoing specification, the disclosure has been provided with reference to specific exemplary embodiments thereof. It will be evident that various modifications may be made thereto without departing from the broader spirit and scope as set forth in the following claims. The specification and drawings are, accordingly, to be regarded in an illustrative sense rather than a restrictive sense.

What is claimed is:

1. An apparatus, comprising:
an air filter;
a housing to hold the air filter inside the housing, the housing having at least a first vent to allow air to enter inside the housing through the air filter and at least a second vent to allow air to exit from inside of the housing, the housing to prevent exhaled air from going through the air filter;
two plugs coupled with the housing and adapted to be plugged into nostrils of a person to provide air passage between respective nasal passages of the person and inside of the housing; and
two hooks coupled with the housing and adapted to be supported by ears of the person while the plugs are plugged into the nostrils of the person.

2. The apparatus of claim 1, wherein the housing defines:
a first compartment defining an interior space connected to interiors of the plugs to provide the air passages; and
a second compartment to hold the air filter between the at least first vent and the first compartment.

3. The apparatus of claim 2, wherein the housing comprises:
a first wall to separate the first compartment and the second compartment; and
a first valve disposed on the first wall to prevent air flow between the first compartment and the second compartment when the person exhales and to allow air flow between the first compartment and the second compartment when the person inhales.

4. The apparatus of claim 3, wherein the housing further defines:
at least one third compartment defining at least one interior space connected to openings of the at least one second vent.

5. The apparatus of claim 4, wherein the housing further comprises:
at least one second wall to separate the first compartment and the at least one third compartment; and
at least one second valve disposed on the at least one second wall to prevent air flow between the first compartment and the at least one third compartment when the person inhales and to allow air flow between the first compartment and the at least one third compartment when the person exhales.

6. The apparatus of claim 2, wherein the air filter comprises a pre-filter and a HEPA accordion filter.

7. The apparatus of claim 6, wherein the air filter further comprises a carbon filter disposed between the pre-filter and the HEPA accordion filter.

8. The apparatus of claim 2, wherein the housing comprises a cover; and the air filter is replaceable when the cover is open.

9. The apparatus of claim 1, further comprising:
an electronic device to provide audio signals; and
earphones coupled to the electronic device and supported by the hooks.

10. The apparatus of claim 9, wherein the electronic device comprises a music player.

11. An apparatus, comprising:
a housing to hold an air filter inside the housing; and
two plugs disposed on the housing and adapted to be plugged into nostrils of a person to provide air passages between respective nasal passages of the person and outside of the housing through the air filter held inside the housing.

12. The apparatus of claim 11, wherein the housing is configured to be disposed between the nose of the person and the mouth of the person.

13. The apparatus of claim 12, further comprises ear hooks coupled to the housing to provide support via the ears of the person.

14. The apparatus of claim 12, wherein the housing does not cover above the nose of the person from outside of the nose.

15. The apparatus of claim 12, wherein the housing does not cover the mouth of the person when the person opens the mouth.

16. The apparatus of claim 11, wherein each plug of the two plugs comprises:
a tube to provide an air passage from a nasal passage of the person to inside of the housing; and
compressible foam to air-tightly engage the plug with interior of a nostril of the person.

17. The apparatus of claim 11, wherein the housing comprises an upper exterior wall and a lower exterior wall; the plugs are disposed over the upper exterior wall; and when the plugs are inside the nostril of the person, the upper exterior wall is under the nose of the person and the lower exterior wall above the mouth opening of the person.

18. The apparatus of claim 11, wherein the housing comprises a removable cover, which when removed provides an opening to service the air filter.

19. The apparatus of claim 18, wherein the housing comprises separate exhalation vents and inhalation vents.

20. The apparatus of claim 11, further comprising:
an air filter housed within the housing.
